(19) United States
(12) Patent Application Publication
(10) Pub. No.: US 2007/0088285 A1
(43) Pub. Date: Apr. 19, 2007

(54) Syringe marking template

(75) Inventors: Fraser R. Sharp, Vancouver (CA); Laurel A. Novacek, Vancouver (CA)

Correspondence Address: NIXON & VANDERHYE, PC, 901 NORTH GLEBE ROAD, 11TH FLOOR, ARLINGTON, VA, 22203, US

(73) Assignee: Inviro Medical Devices, Ltd., Bridgetown (BB)

(21) Appl. No.: 11/518,322

(22) Filed: Sep. 11, 2006

Related U.S. Application Data
(60) Provisional application No. 60/715,590, filed on Sep. 12, 2005.

(51) Int. Cl. A61M 5/00

(52) U.S. Cl. 604/187

(57) ABSTRACT

A syringe includes an elongated syringe barrel formed of a transparent material; a cannula adjacent one end of the barrel for transmission of fluid into our out of the barrel; a plunger disposed in the barrel and extending through an opposite end of the barrel from the one end; index markings along one side of the barrel for use in identifying various dosage amounts; and a collar moveable along the barrel facilitating indication of a custom marking of a desired dosage amount on the barrel.

Priority is hereby claimed from U.S. Provisional Application Ser. No. 60/715,590, filed Sep. 12, 2005, the entirety of which is hereby incorporated by reference.

### TECHNICAL FIELD

The present invention relates generally to syringes and particularly to a syringe marking system which may include a template for visualization and/or application of custom markings to the syringe to assist in reducing errors inherent in the visualization of often very small index markings, and the associated difficulties inherent in accurately aligning the plunger with these markings, to ensure the volumetric accuracy in delivering the required dosages in either single or repeated used of a syringe. The same principles can be applied to other non-medical syringe uses, for example, in industrial processes.

### BACKGROUND AND SUMMARY OF THE INVENTION

Patients with chronic illnesses such as diabetes who require regular injection will, sometimes against medical advice, use a hypodermic syringe for injecting themselves on a number of occasions rather than the single syringe use which is generally recommended. Fortunately, this practice if it is carried out with care, does not result in an unusual number of infections. This may be the reason why it has been estimated that diabetic patients on average will use the same syringe for multiple injections.

For patients with a stable disease, it is often necessary to inject with a standard dose of insulin on numerous occasions. Because an insulin syringe is physically small (1 ml. or less), reading the index markings both linear and numerical can be difficult. Each time the syringe is filled, there is a possibility that a misreading of the index markings will occur and the wrong dose delivered. This situation could be alleviated if a syringe barrel is custom marked with a line of greater thickness or length corresponding to the much smaller index markings already on the syringe and at the level of the dose required. Large easily read numerical marks can also be applied by hand to identify the applied linear index marking. Assuming each syringe is customized for a specific dose each time insulin is drawn into that syringe, the plunger stopper may be aligned with this more easily seen line applied by hand to the barrel rather than the much smaller index markings printed on the barrel during manufacture. As the index markings take up approximately one half of the circumference of the barrel there is ample room on the remainder of the barrel to draw a custom marking line. It should be emphasized that enhancing the ability to accurately draw up the medication dose repeatedly to the same level should not be confused with the recommendations that a single use syringe should not be used multiple times. However, the reality is that diabetic patients typically use the same syringe for multiple injections and any method of simplifying the accurate drawing up of a dose of medication would presumably be welcome, reducing the risk of error and improving safety for the diabetic patient.

It should also be noted that diabetic patients will frequently have poor eyesight, due to diabetic retinopathy and visualization of the index markings on the syringe barrel may be very difficult for them. If a much more easily visualized mark or line is applied to the barrel, even possibly by a third party with better eyesight, this would allow patients to draw up their own medication repeatedly and more accurately even in the circumstance of poor eyesight.

U.S. Pat. No. 6,315,760 issued Nov. 13, 2001, of common assignee herewith, discloses a syringe having a white or contrasting write-on stripe applied to the syringe barrel to enhance the visualization of index markings and also to allow writing on this stripe. It will be appreciated that this contrasting stripe will enhance the ability to apply dose markings. However, not all syringe barrels have a contrasting stripe and it may be desirable to apply a custom dose line with a light colored or other contrasting colored marking pen. Fluorescent or other enhancements may also be applied and by a mechanical device rather than by hand.

### BRIEF SUMMARY

This disclosure relates to a method of accurately applying custom markings such as a line to the barrel of a syringe to allow accurate filling of the syringe to a predetermined dose or more easily visualizing and identifying existing index markings present on a syringe during filling of the syringe. The accurate filling of a syringe involves performing visual and mechanical tasks simultaneously often with very small syringes. This can be difficult particularly for patients required to self inject who may have visual, cognitive or motor impairment. Accordingly, a type of template or dosage marker may be employed to indicate or to enable application of additional custom markings to simplify and enhance accuracy and speed of drawing up the desired dose of medication by the patient or caregiver. In one embodiment, the refractive index of a plastic or other template material may also provide magnification of the index markings enabling accurate visualization of the desired dose and permitting the easier application of custom markings to the barrel at the desired dose level,

Preferred components of the present invention include a dosage collar with certain properties which can be applied to the syringe and moved along the axis and aligned with the index markings present on the conventional syringe. The collar may contain an index line itself or alternatively one of the two borders of the collar can be used to align with the index markings. If desired a pen with particular characteristics can also be used to allow marking on the barrel, (or any suitable writing instrument if the write-on stripe is present). The template is movable axially along the barrel and then fixed to enable the pen to apply the custom mark. The collar therefore is movable and may be fixed at a selected location along the barrel to allow it to be used as a dosage marker or template for marking the barrel. The template may be formed of elastomeric or flexibly resilient material or with various clamping and release methodologies. The template can be set at a selected dose location along the barrel using the index markings or borders of the collar or cutouts in the collar. The template fits snugly about the barrel and, once positioned, resists movement, thereby enabling a pen or other writing instruments to be placed against the template for use as a guide for drawing the custom line or other mark on the barrel as described above.

The collar may also have a cutout on either side to allow visualization of the index markings or, alternatively, to permit the tip of the pen to be inserted into the cutout for drawing the marking on the barrel. In one embodiment some magnification using the refractive index of the material of the template, e.g. plastic, may be desirable to enhance visualization of the index markings

The collar or template may be permanently applied to each syringe or moveable from one syringe to another. In addition to referencing the index markings, the template may also use other features on the syringe as a baseline for measuring dosage, such as the finger grips on the syringe barrel.

Accordingly, in one aspect, the invention relates to a syringe comprising: an elongated syringe barrel formed of a transparent material; a cannula adjacent one end of said barrel for transmission of fluid into our out of said barrel; a plunger disposed in said barrel and extending through an opposite end of said barrel from said one end; index markings along one side of said barrel for use in identifying various dosage amounts; a contrast stripe along another side of the barrel opposite said one side, said contrast stripe forming a marking area along said index markings; a collar moveable along said barrel facilitating indication of or custom marking of a desired dosage amount on said contrast stripe.

In another aspect, the invention relates to a syringe comprising: an elongated syringe barrel formed of a transparent material; a cannula adjacent one end of said barrel for transmission of fluid into our out of said barrel; a plunger disposed in said barrel and extending through an opposite end of said barrel from said one end; index markings along one side of said barrel for use in identifying various dosage amounts; and a dosage collar moveable along said barrel facilitating indication of or custom marking of a desired dosage amount on said barrel.

### BRIEF DESCRIPTION OF THE DRAWINGS

FIG. 1 is a side elevational view of a known syringe;

FIG. 2 is a side elevational view of the syringe with a marking template applied;

FIG. 3 is a perspective view of a representative example of a marking template for use with the syringe;

FIG. 4 is a top plan view of a marking template in accordance with another aspect of the present invention;

FIG. 5 is a perspective view of the template of FIG. 4;

FIG. 6 is a partial perspective view of a marking template in accordance with another aspect of the invention;

FIG. 7 is a view similar to FIG. 1 with a marking template applied, and using a magnifying aspect of the present invention;

FIG. 8 is a view similar to FIG. 1 with the marking applied;

FIG. 9 is a view similar to FIG. 6 illustrating an alternative template in accordance with another example of the present invention; and

FIG. 10 is a view similar to FIG. 7 but illustrating still another marking template; and

FIG. 11 is a perspective view illustrating a marking template sleeve of a different size than shown in FIG. 10.

### DETAILED DESCRIPTION OF THE INVENTION

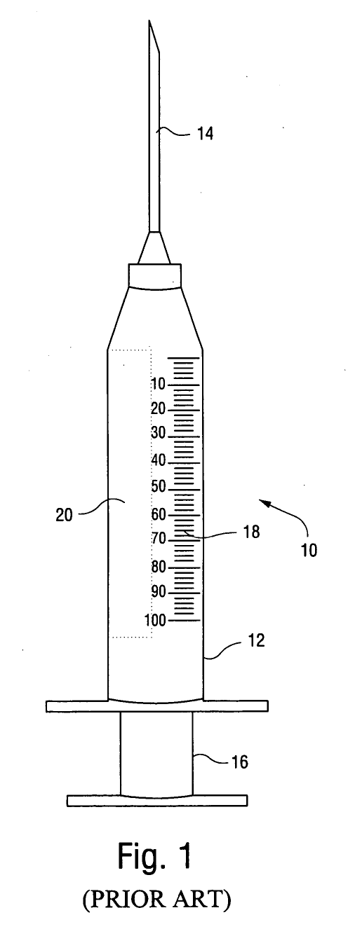

Referring to the drawings particularly to FIG. 1, there is illustrated a typical syringe generally designated 10 including a barrel 12, a needle or cannula 14 on the distal end of the barrel and a plunger 16 slidable within the barrel. Syringe 10 is of a conventional construction and includes index markings 18 on the barrel as well as a contrasting color marking or stripe 20 coextensive with the index markings 18 along the opposite side of the barrel from the index markings. The contrast marking or stripe 20 is disclosed in U.S. Pat. No. 6,315,760, incorporated herein by reference.

As the above discussion indicates, the index markings 18 particularly on small diameter syringes e.g. 1 ml., 0.5 ml. or 0.3 ml. are very difficult to see and it is therefore difficult align the plunger 16 accurately to ensure accurate filling with medicament and therefore correct dose delivery to the patient.

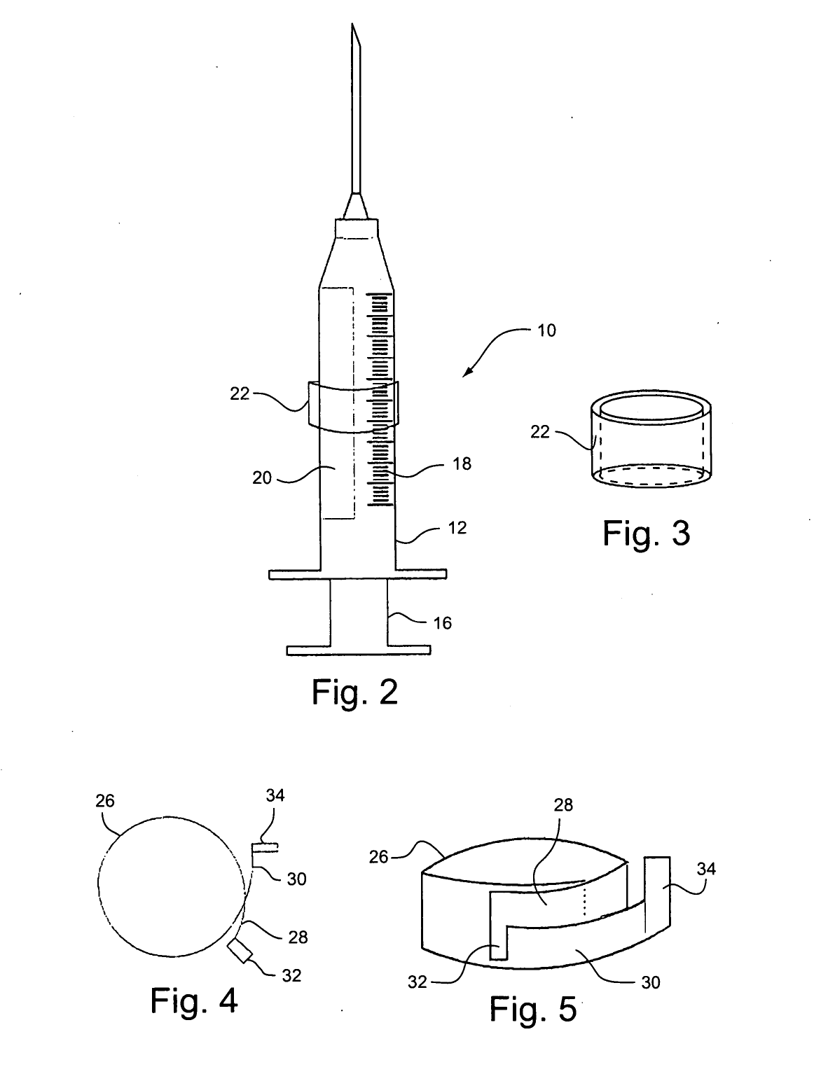

Referring to FIG. 2, there is provided a custom dosage template or collar 22 constructed in accordance with one aspect of the present invention, and that facilitates custom marking of the syringe barrel such that a single or repeated doses of the same magnitude can be withdrawn into the syringe and/or expelled. As indicated in FIG. 3, collar 22 may be a transparent or opaque elastomeric, solid, cylindrical sleeve or ring.

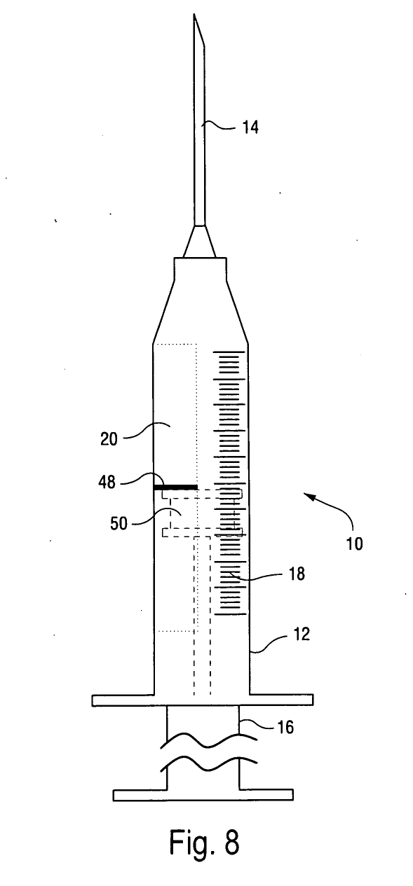

In use, the collar 22 is slipped over the end of the syringe (or may be prepositioned at, for example, the finger hold end of the syringe during manufacture) and one or the other of opposite edges of the collar is aligned with the index marking identifying a desired dose. With that alignment, a marking pencil (or other device capable of making a linear mark on the barrel) may be drawn about the barrel, for example, in the area of the contrasting stripe 20 such that the mark can be readily seen even by individuals with impaired eyesight. A numerical reference can also, if desired, be applied to the barrel to identify the line. Because of the necessarily small preprinted index markings, the custom numerical mark can easily be 2 to 3 times larger, or more, allowing comparatively much easier visualization. With the syringe barrel thus custom marked, the plunger bung 50 (see FIG. 8) may be aligned with the custom marking and thereby the syringe may be used to repeatedly withdraw and/or expel multiple identical doses of the medicament. The collar may be retained on or removed from the syringe barrel and may be used not only to apply a custom marking index line as discussed above, but also used itself as a marker

Referring to FIGS. 4 and 5, there is illustrated a further form of collar. In this form, a flexible collar 26 is formed of a plastic or elastic material resiliently biased to a diameter smaller than the outer diameter of the syringe barrel. The collar 26 includes a pair of overlapping upper and lower legs 28 and 30, each terminating in finger presses 32 and 34 respectively. By pressing the finger presses 32 and 34 toward one another thereby enlarging the diameter of the collar 26, the collar may be disposed about the syringe barrel with one or the other of the edges aligned with the appropriate index marking. By releasing the finger presses 32 and 34, the collar 26 is biased for frictional retention on the syringe barrel. The custom marking may then be applied to the barrel as previously noted, or the collar itself may be used as an index marker.

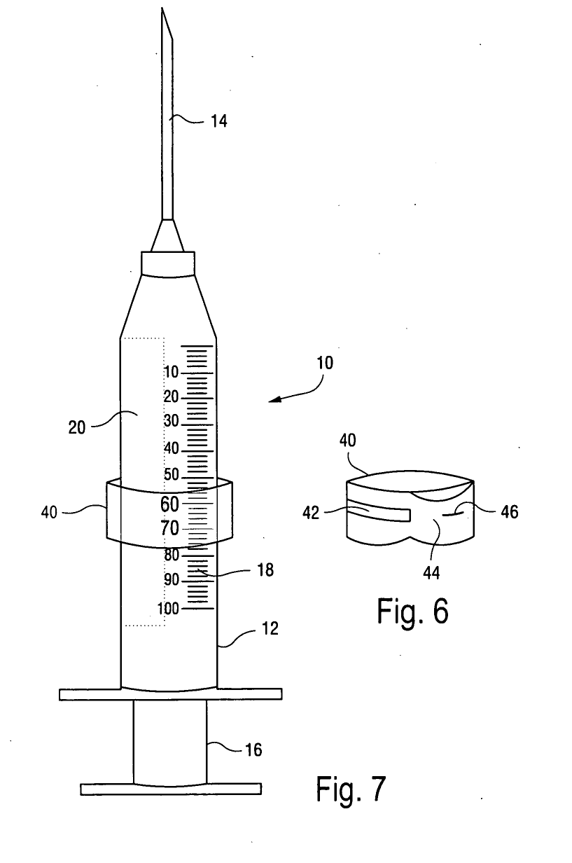

Referring to FIG. 6, a collar 40 may have a slot 42 formed in part about its circumference. When the collar 40 is disposed about the barrel and the slot 42 registers with the desired index marking indicating the appropriate dosage, the barrel can be marked using a marking pencil extending into the slot 42. Alternatively, the collar may be left in the desired location, with the slot used as a marker. Also, as illustrated in FIG. 7, the inner and outer curvature of a portion of collar 40 may be configured to provide magnification. For example, a convex projection along this outside surface of the collar having a different radius to inside surface may provide magnification of the index markings such as illustrated in FIG. 7. The magnified index markings of course appear as the collar is displaced along the syringe barrel. The syringe barrel can be custom marked when the collar is aligned on either side edge with the appropriate index marking or custom marked when collar 40 is aligned with the index marking using an indicator 46 on the magnifying surface of the collar itself.

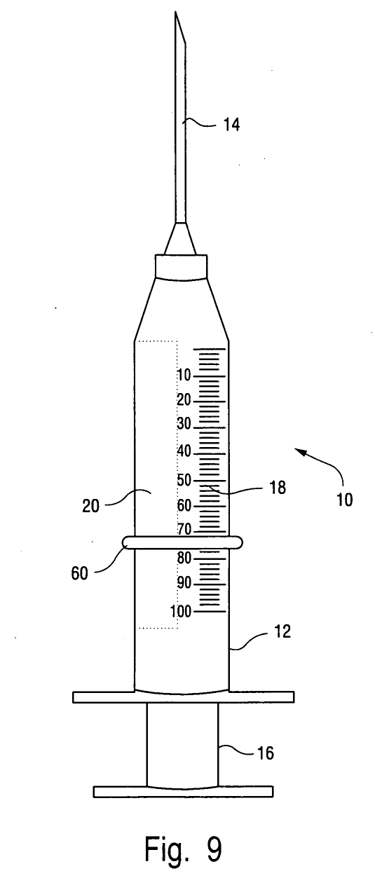

In FIG. 8, a custom marking is illustrated at a line 48, in this instance drawn along the contrasting stripe area. The forwarded edge of the bung 50 of the plunger 16 is illustrated aligned with the custom marking 48. 100281 Referring now to FIG. 9, the syringe may have a collar in the form of an elastomeric O-ring 60. By rolling the O-ring 60 along the length of the barrel and locating it in alignment with a predetermined index marking, the barrel may be custom marked to provide multiple identical dosages for multiple usage of the syringe as previously explained, or the O-ring itself may be used as the marker.

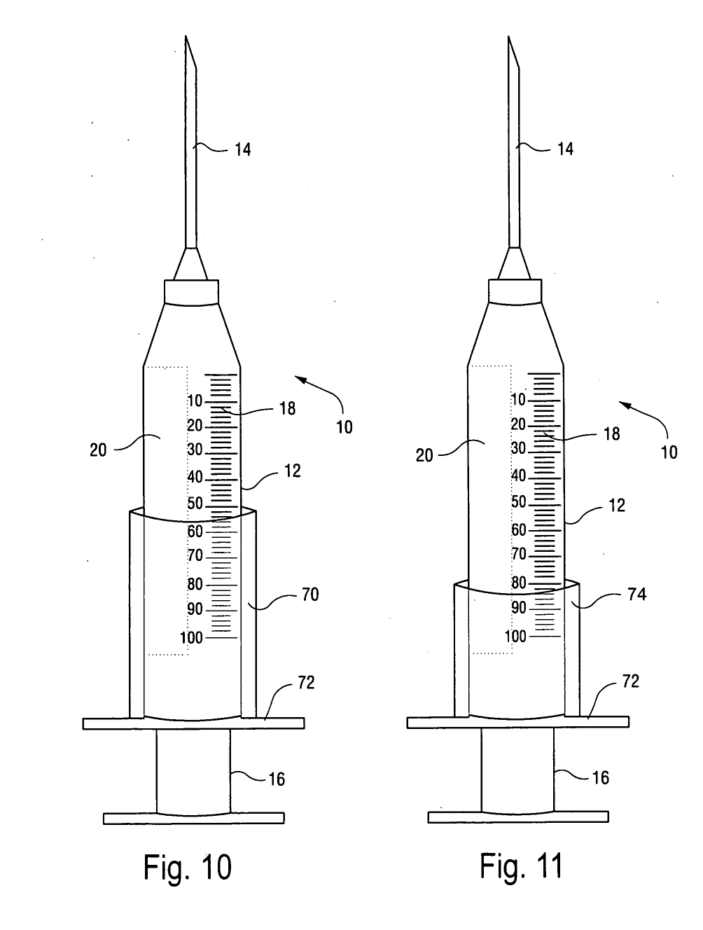

Referring now to FIG. 10, one of a selected number of sleeves 70 of different lengths may be provided about the barrel with one end of the selected sleeve 70 bearing against the finger press 72 at the proximal end of the syringe barrel. The opposite end of the sleeve may register with a predetermined dose marking along the index markings 18. For example, the sleeve may have a length extending from the finger press 72 to the index marking “54”. With the sleeve disposed about the syringe barrel, the barrel can be custom marked on the stripe 20 at the “54” index marking. As illustrated in FIG. 11, another sleeve 74 may be provided having a different length than the length of sleeve 70 so that, when its proximal end butts finger press 72, its distal end registers with different index marking, for example, “82”. Consequently, when the custom marking is applied as in FIG. 11, a different dose than the dose indicated in FIG. 10, may be repeatedly withdrawn or expelled. It will be appreciated that a set or series of sleeves may be provided of different lengths such that the distal end of the sleeves may register with predetermined markings whereby custom markings can be applied to the barrel to indicate predetermined different doses, or the distal ends of the sleeves may be used as markers without custom markers applied to the barrel.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the disclosure.

What is claimed is:

1. A syringe comprising:
an elongated syringe barrel formed of a transparent material;
a cannula adjacent one end of said barrel for transmission of fluid into our out of said barrel;
a plunger disposed in said barrel and extending through an opposite end of said barrel from said one end;
index markings along one side of said barrel for use in identifying various dosage amounts;
a contrast stripe along another side of the barrel opposite said one side, said contrast stripe forming a marking area along said index markings;
a collar moveable along said barrel facilitating indication of or custom marking of a desired dosage amount on said contrast stripe.

2. The syringe of claim 1, wherein said collar comprises a transparent or opaque sleeve having opposite edges, either of said edges alignable with one of said index marking corresponding to said desired dosage.

3. The syringe of claim 2 wherein said sleeve is transparent and formed with a convex projection along an outer surface thereof providing a magnification of underlying ones of said index markings.

4. The syringe of claim 1 wherein said collar comprises a solid cylindrical sleeve with a linear slot extending about a peripheral portion thereof, said slot sized to permit easier identification or visualization and/or a marking device to penetrate said slot and mark said barrel or contrast stripe to indicate said desired dosage.

5. The syringe of claim 4 wherein a linear mark is provided on said device, axially aligned with and circumferentially spaced from said slot to thereby enable accurate dosage indication and/or marking of said contrast stripe.

6. The syringe of claim 1 wherein said collar comprises an O-ring.

7. The syringe of claim 1 wherein said collar comprises an elongated sleeve, one end of which is adapted to engage a finger press at a proximal end of said syringe barrel, said elongated sleeve having a length such that an opposite end thereof aligns with one of said index markings corresponding to said desired dosage.

8. The syringe of claim 1 wherein said marking collar is constructed of resilient material and formed with a pair of upper and lower overlapping legs, each terminating in a finger press enabling enlargement of the collar and resilient attachment to said barrel.

9. A syringe comprising:
an elongated syringe barrel formed of a transparent material;
a cannula adjacent one end of said barrel for transmission of fluid into our out of said barrel;
a plunger disposed in said barrel and extending through an opposite end of said barrel from said one end;
index markings along one side of said barrel for use in identifying various dosage amounts; and
a dosage collar moveable along said barrel facilitating indication of or custom marking of a desired dosage amount on said barrel.

10. The syringe of claim 9, wherein said collar comprises a transparent or opaque sleeve having opposite edges, either of said edges alignable with one of said index marking corresponding to said desired dosage.

11. The syringe of claim 10 wherein said sleeve is transparent and formed with a convex projection along an outer surface thereof providing a magnification of underlying ones of said index markings.

12. The syringe of claim 9 wherein said marking collar comprises a solid cylindrical sleeve with a linear slot extending about a peripheral portion thereof, said slot sized to permit easier identification of or visualization and/or a marking device to penetrate said slot and mark said desired dosage on said barrel.

13. The syringe of claim 12 wherein a linear mark is provided on said device, axially aligned with and circumferentially spaced from said slot to thereby enable accurate dosage indication and/or marking of said barrel.

14. The syringe of claim 12 wherein said collar comprises an O-ring.

15. The syringe of claim 9 wherein said collar is constructed of resilient material and formed with a pair of upper and lower overlapping legs, each terminating in a finger press enabling enlargement of the collar and resilient attachment to said barrel
